(19) United States
(12) Patent Application Publication
(10) Pub. No.: US 2025/0102487 A1
(43) Pub. Date: Mar. 27, 2025

(54) Water Sensor Buoy with Removable Sensor Package

(71) Applicant: Precision Measurement Engineering, Inc., Vista, CA (US)

(72) Inventor: Kristin Michelle Elliott, San Marcos, CA (US)

(21) Appl. No.: 18/474,600

(22) Filed: Sep. 26, 2023

(51) Int. Cl. G01N 33/18; B63B 22/00; G01N 21/64

(52) U.S. Cl. CPC G01N 33/1886; B63B 22/00; G01N 21/64; B63B 2022/006; B63B 2211/02

(57) ABSTRACT

A water sensor buoy for use in a water environment including a main buoy assembly and a sensor package. The assembly includes a buoy hull, a data processing and control unit, a data transmission unit, and a power source. The sensor package is in removable engagement with the assembly. The sensor package includes a sensor housing in removable engagement with the buoy hull, and a water sensor in removable electronic communication with the data processing and control unit and the power source, and configured to sense an attribute of the water environment. The buoy has a watertight seal between the buoy hull and sensor housing, and an electrical conduit. The conduit is engageable with the data processing and control unit and the power source with the sensor housing being engaged with the buoy hull, and disengageable upon the sensor housing being disengaged from the buoy hull.

### CROSS-REFERENCE TO RELATED APPLICATIONS

Not Applicable

### STATEMENT RE: FEDERALLY SPONSORED RESEARCH/DEVELOPMENT

Not Applicable

### BACKGROUND

There are a variety of attributes of water or aqueous environments that are desirable to be measured and monitored. Such water environments may range from oceans, lakes, reservoirs, holding ponds, to pools. Various sensors or probes may be deployed in such water environments. For example, optical fluorescence sensors are single wavelength in situ fluorescence and turbidity probes used for monitoring various water quality parameters such as chlorophyll-a concentration and turbidity in aquatic systems. Acoustic backscatter sensors are used to measure the concentration and size distribution of suspended particles or sediments in water. Photosynthetically active radiation (PAR) sensors work by measuring the intensity of light that is used by plants for photosynthesis. There are a variety of types of conductivity sensors available that are used for measuring the electrical conductivity of water, which is related to the concentration of dissolved materials in the water, such as salts. Hydrophones are underwater microphones that are used to detect and measure sound waves in water. There are a variety of pH sensors for measuring the acidity or alkalinity of a solution.

Such water sensors may be deployed in a mobile manner, such as installed in a float or buoy. These water sensor buoys would have a hull or housing that contains a sealable chamber or inflatable bladder for floatation on the water surface. The water sensor would typically be installed at the bottom side of the buoy to be exposed to the water environment when the buoy is floating. Sensor data may then be collected and processed by water sensor and associated electronics. In some applications, sensed data may also be wirelessly transmitted by the water sensor buoy so as to enable retrieval of real-time or current data. Additionally, location data may be generated, such as GPS data, which may be correlated to the sensor data. An array of these water sensor buoys may be utilized for a desired coverage area.

Servicing, inspection and maintenance of such water sensor buoys is of concern. The water sensors have an exterior sensor surface that is exposed to the water environment. Depending upon the sensor type, in some instances this may take the form of a thin membrane or foil, and in others, this may be a small window or sheet of translucent glass, as example. The exterior sensor surface may become fouled with accumulation particulates disposed upon the exterior sensor surface. This may be due to adhesion or deposition of material in the water environment and growth of algae and other living organisms. Fouling is an issue associated with all sensor instrumentation deployed in water environments that causes a loss in sensitivity and reproducibility of the water sensor data, thus requiring frequent re-calibration and/or producing erroneous results.

In view of the foregoing, there is a need in the art for an improved apparatus and/or method of deploying and maintaining water sensor buoys.

### BRIEF SUMMARY

According to an aspect of the invention, there is provided a water sensor buoy for use in a water environment. The water sensor buoy includes a main buoy assembly sized and configured to float in a water environment. The main buoy assembly includes a buoy hull sized and configured to float in the water environment, a data processing and control unit, a data transmission unit, and a power source. The data processing and control unit is supported by the buoy hull. The data processing and control unit is sized and configured to generate a data signal upon receipt of sensor data from a water sensor. The data transmission unit is supported by the buoy hull and is in electronic communication with the data processing and control unit. The data transmission unit is sized and configured to wirelessly transmit a data signal generated by the data processing and control unit. The power source is supported by the buoy hull and is in electrical communication with the data processing and control unit and the data transmission unit. The water sensor buoy further includes a sensor package in removable engagement with the main buoy assembly. The sensor package includes a sensor housing in removable engagement with the buoy hull and a water sensor. The water sensor is disposed in the sensor housing. The water sensor has a sensor casing, a sensing element attached to the sensor casing, and sensor electronics in electrical communication with the sensing element. The sensor casing is attached to the sensor housing. The water sensor is in removable electronic communication with the data processing and control unit and the power source. The water sensor is sized and configured to sense an attribute of the water environment and generate sensor data for receipt by the data processing and control unit. The water sensor is electronically disengageable from the data processing and control unit and the power source upon the sensor housing being disengaged from the buoy hull. The water sensor buoy further includes a watertight seal formed between the buoy hull and sensor housing with the sensor housing engaged with the buoy hull. The water sensor buoy further includes an electrical conduit disposed through the watertight seal with the sensor housing engaged with the buoy hull. The electrical conduit is electrically connected to the sensor electronics. The electrical conduit is electronically engageable with the data processing and control unit and the power source with the sensor housing being engaged with the buoy hull. The electrical conduit is electronically disengageable from the data processing and control unit and the power source upon the sensor housing being disengaged from the buoy hull.

According to various embodiments, the water sensor buoy may further include an O-ring disposed between the buoy hull and the sensor housing for forming the watertight seal. The sensor housing may have a sensor cover sized and configured to engage the buoy hull, and the watertight seal may be disposed between the sensor cover and the buoy hull with the electrical conduit is disposed through the sensor cover. The sensor cover may be sized and configured to be circumferentially received by the buoy hull for engagement of the buoy hull and the sensor housing. The sensor housing may have an exit port, and the electrical conduit may be disposed through the exit port. The electronic conduit includes an exit port coupling sized and configured to electronically engage and disengage with the data processing and control unit and the power source. The buoy hull may include a chamber, and the chamber may be watertight upon the buoy hull being engaged with the sensor housing. The water sensor may be a pH sensor, an optical fluorescence sensor, or a hydrophone.

According to another aspect of the invention, there is provided a method of using a water sensor buoy for use in a water environment. The method includes the step of floating the water sensor buoy in the water environment. The water sensor buoy includes a main buoy assembly sized and configured to float in a water environment, a sensor package, a watertight seal, and an electrical conduit. The main buoy assembly includes a buoy hull sized and configured to float in the water environment. The main buoy assembly further includes a data processing and control unit supported by the buoy hull. The data processing and control unit is sized and configured to generate a data signal upon receipt of sensor data from a water sensor. The main buoy assembly further includes a data transmission unit supported by the buoy hull and is in electronic communication with the data processing and control unit. The data transmission unit is sized and configured to wirelessly transmit a data signal generated by the data processing and control unit. The main buoy assembly further includes a power source supported by the buoy hull and is in electrical communication with the data processing and control unit and the data transmission unit. The water sensor buoy further includes a sensor package being in removable engagement with the main buoy assembly. The sensor package includes a sensor housing in removable engagement with the buoy hull, and a water sensor. The water sensor disposed in the sensor housing. The water sensor has a sensor casing, a sensing element attached to the sensor casing, and sensor electronics in electrical communication with the sensing element. The sensor casing is attached to the sensor housing. The water sensor is in removable electronic communication with the data processing and control unit and the power source. The water sensor is sized and configured to sense an attribute of the water environment and generate sensor data for receipt by the data processing and control unit. The water sensor is electronically disengageable from the data processing and control unit and the power source upon the sensor housing being disengaged from the buoy hull. The water sensor buoy further includes a watertight seal formed between the buoy hull and sensor housing with the sensor housing engaged with the buoy hull. The water sensor buoy further includes an electrical conduit disposed through the watertight seal with the sensor housing engaged with the buoy hull. The electrical conduit is electrically connected to the sensor electronics. The electrical conduit is electronically engageable to the data processing and control unit and the power source with the sensor housing being engaged with the buoy hull. The electrical conduit is electronically disengageable from the data processing and control unit and the power source upon the sensor housing being disengaged from the buoy hull. The method may further include removing the water sensor buoy from the water environment. The method may further include disengaging the sensor housing from the buoy hull. The method may further include electronically disengaging the electrical conduit from the data processing and control unit and the power source.

According to an aspect of the invention, there is provided a water sensor buoy for use in a water environment. The water sensor buoy includes a buoy hull sized and configured to float in the water environment. The water sensor buoy further includes a data processing and control unit supported by the buoy hull. The data processing and control unit is sized and configured to generate a data signal upon receipt of sensor data from a water sensor. The water sensor buoy further includes a data transmission unit supported by the buoy hull and is in electronic communication with the data processing and control unit. The data transmission unit is sized and configured to wirelessly transmit a data signal generated by the data processing and control unit. The water sensor buoy further includes a power source supported by the buoy hull and is in electrical communication with the data processing and control unit and the data transmission unit. The water sensor buoy further includes a water sensor supported by the buoy hull. The water sensor includes a sensing element and an exterior sensor surface facing away from the sensing element. The sensing element is in electronic communication with the data processing and control unit and the power source. The sensing element is sized and configured to sense an attribute of the water environment and generate sensor data for receipt by the data processing and control unit. The exterior sensor surface is exposed to the water environment upon the water sensor buoy being placed in the water environment. The water sensor buoy further includes a wiper wheel rotatably supported by the buoy hull. The water sensor buoy further includes a wiper attached to the wiper wheel. The wiper is sized and configured to slidably engage the exterior sensor surface upon rotation of the wiper wheel for removal of particulates disposed upon the exterior sensor surface. The water sensor buoy further includes a water pump being in electrical communication with the power source. The water sensor buoy further includes a water jet outlet in fluid communication with the water pump. The water jet outlet is sized and configured to direct a water stream at the exterior sensor surface for removal of particulates disposed upon the exterior sensor surface.

According to various embodiments, the water sensor buoy may further include a wheel motor electrically connected to the power source and engaged with the wiper wheel. The wheel motor is sized and configured to rotate the wiper wheel to move the wiper relative to the exterior sensor surface. The water sensor buoy may further include a sensor housing engaged with the buoy hull. The water sensor may be attached to the sensor housing. The wiper wheel may be rotatably attached to the sensor housing, and the water pump may be attached to the sensor housing. The wiper wheel may include a radially extending slot, and the wiper is disposed in the slot. The wiper may be press-fit attached to the wiper wheel in the slot. The wiper may include a brush. The wiper may include a wiper arm and the brush extends from the wiper arm. The wiper wheel may include a radially extending slot, and the wiper arm is disposed in the slot. The wiper may be press-fit attached to the wiper wheel. The water sensor may be a pH sensor, an optical fluorescence sensor, or a hydrophone. The water sensor may include a sensor foil, and the exterior sensor surface is formed on the sensor foil. The sensor foil may be formed of glass. The sensor foil may be translucent.

According to another aspect of the invention, there is provided a method of operating a water sensor buoy for use in a water environment. The method includes the steps of floating the water sensor buoy in the water environment. The water sensor buoy includes a buoy hull. The water sensor buoy further includes a data processing and control unit supported by the buoy hull. The water sensor buoy further includes a power source supported by the buoy hull and is in electrical communication with the data processing and control unit and the data transmission unit. The water sensor buoy further includes a water sensor supported by the buoy hull. The water sensor includes a sensing element and an exterior sensor surface facing away from the sensing element. The sensing element is in electronic communication with the data processing and control unit and the power source. The sensing element is sized and configured to sense an attribute of the water environment and generate sensor data for receipt by the data processing and control unit. The exterior sensor surface is exposed to the water environment upon the water sensor buoy being placed in the water environment. The water sensor buoy further includes a wiper wheel rotatably supported by the buoy hull. The water sensor buoy further includes a wiper attached to the wiper wheel. The water sensor buoy further includes a water pump being in electrical communication with the power source. The water sensor buoy further includes a water jet outlet in fluid communication with the water pump. The method further includes the step of rotating the wiper wheel to move the wiper to slidably engage the exterior sensor surface for removal of particulates disposed upon the exterior sensor surface. The method further includes the step of directing a water stream from the water jet outlet at the exterior sensor surface for removal of particulates disposed upon the exterior sensor surface.

According to various embodiments, the method may further include pumping a fluid from the water environment by the water pump to the water jet outlet. The method may further include generating a data signal by the data processing and control unit upon receipt of sensor data from the water sensor. The water sensor buoy may further include a data transmission unit supported by the buoy hull and is in electronic communication with the data processing and control unit, and the method may further include wirelessly transmitting the data signal generated by the data processing and control unit. The wiper may include a brush, and the rotating of the wiper wheel may include slidably engaging the brush against the exterior sensor surface for removal of particulates disposed upon the exterior sensor surface.

The present invention will be best understood by reference to the following detailed description when read in conjunction with the accompanying drawings.

### BRIEF DESCRIPTION OF THE DRAWINGS

These and other features and advantages of the various embodiments disclosed herein will be better understood with respect to the following description and drawings, and in which:

FIG. 1 is an exploded top perspective view of a water sensor buoy with a main buoy assembly and a sensor package according to an aspect of the present invention;

FIG. 2 is an exploded bottom perspective view of the water sensor buoy of FIG. 1;

FIG. 3 is an exploded cross-sectional side view of the water sensor buoy of FIG. 1;

FIG. 4 is an assembled side view of the water sensor buoy of FIG. 1 as symbolically depicted in a water environment;

FIG. 5 is an enlarged symbolic side view of components of the water sensor buoy of FIG. 1;

FIG. 6 is an enlarged exploded top perspective view of the sensor package of FIG. 1;

FIG. 7 is a further exploded top perspective view of a portion of the sensor package of FIG. 6;

FIG. 8 is a further exploded bottom perspective view of a portion of the sensor package of FIG. 6;

FIG. 9 is a top perspective view of a wiper wheel of the sensor package; and

FIG. 10 is a bottom perspective view of a wiper wheel of the sensor package.

Common reference numerals are used throughout the drawings and the detailed description to indicate the same elements.

### DETAILED DESCRIPTION

The above description is given by way of example, and not limitation. Given the above disclosure, one skilled in the art could devise variations that are within the scope and spirit of the invention disclosed herein. Further, the various features of the embodiments disclosed herein can be used alone, or in varying combinations with each other and are not intended to be limited to the specific combination described herein. Thus, the scope of the claims is not to be limited by the illustrated embodiments. It is further understood that the use of relational terms such as top and bottom, first and second, and the like are used solely to distinguish one entity from another without necessarily requiring or implying any actual such relationship or order between such entities.

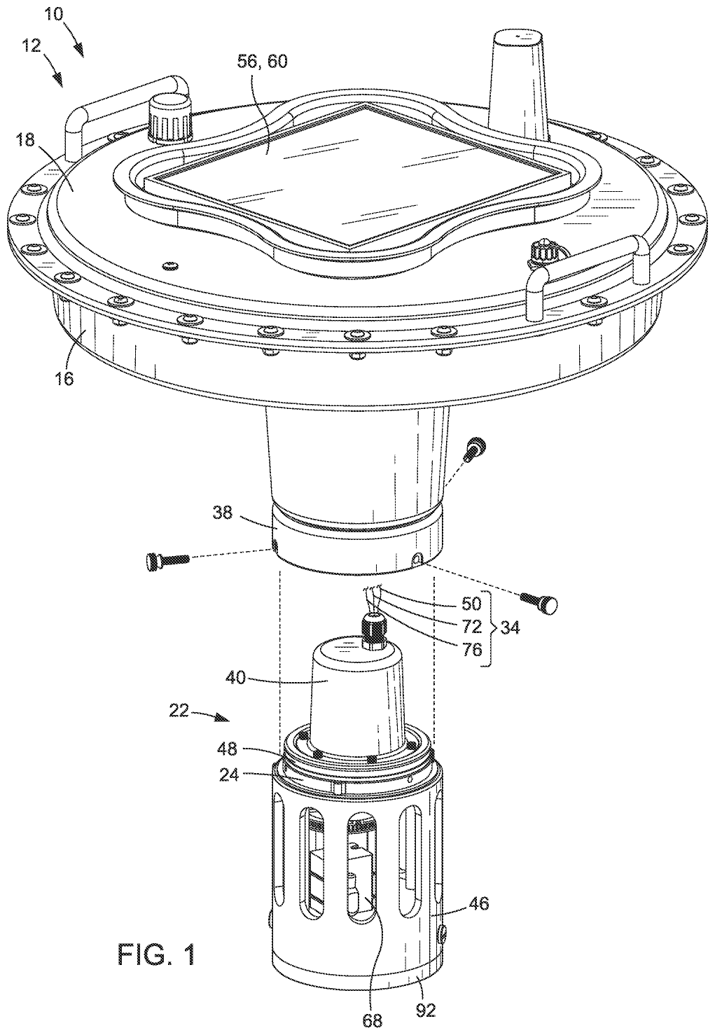

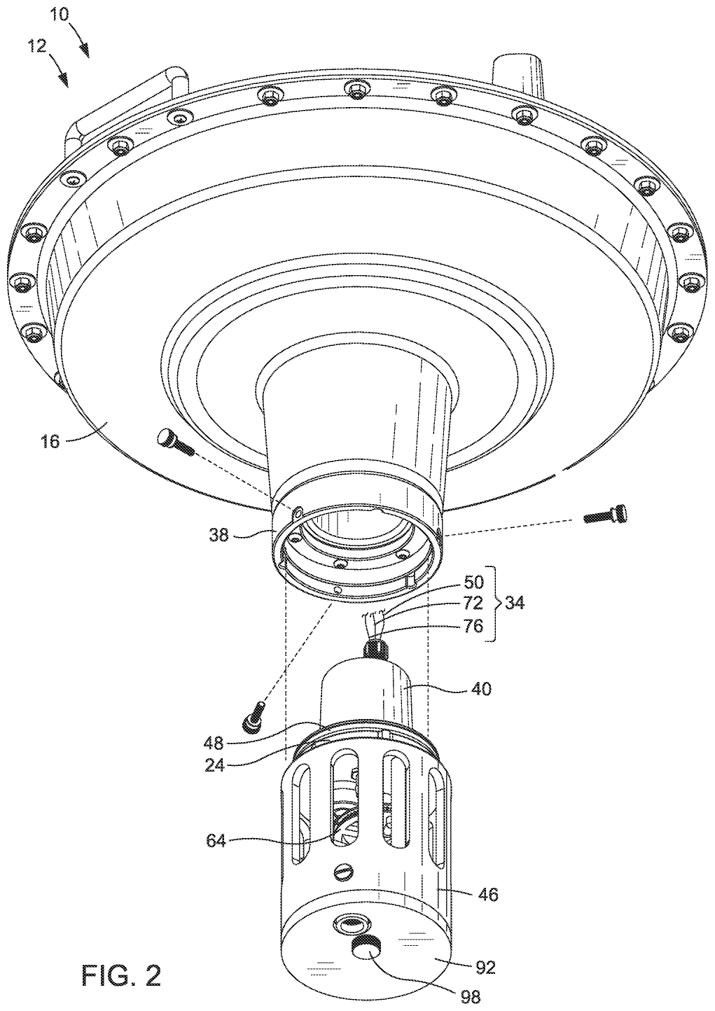

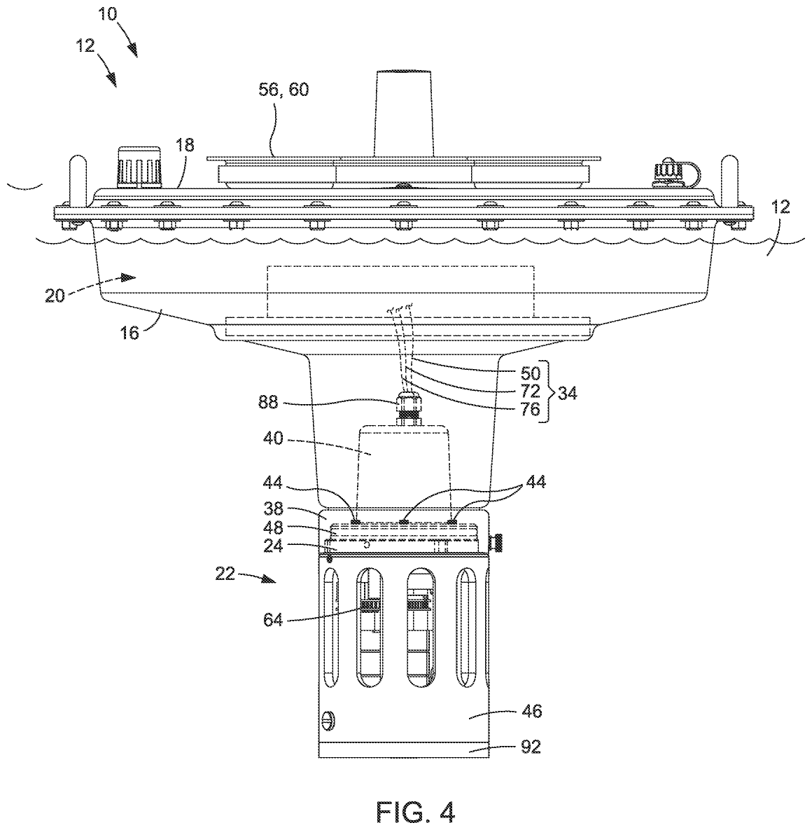

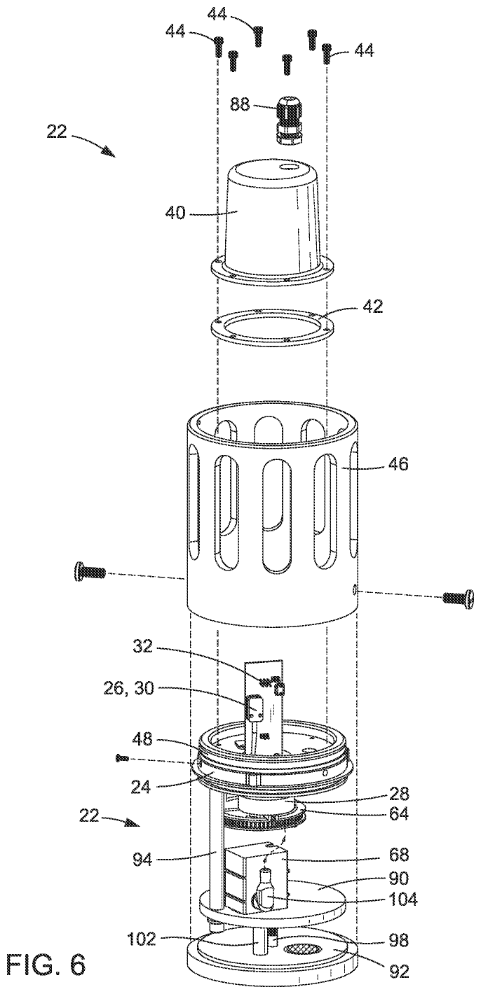

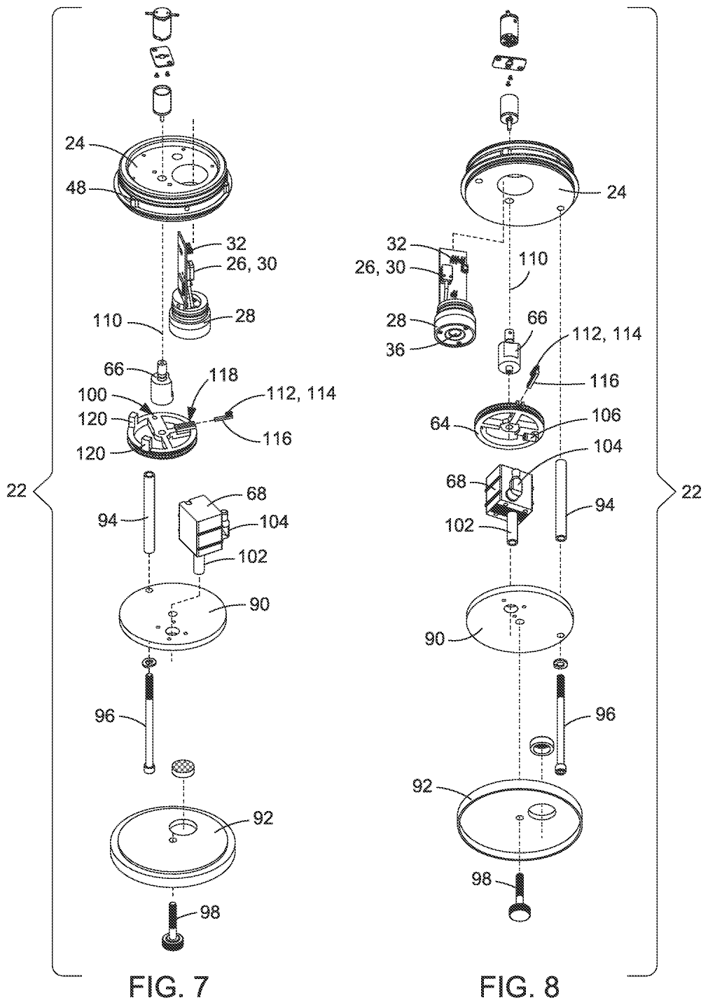

Referring now to FIGS. 1-8, according to an aspect of the present invention, there is provided a water sensor buoy 10 for use in a water environment 12. The water sensor buoy includes a main buoy assembly 14 sized and configured to float in the water environment 12. The main buoy assembly 14 includes a buoy hull 16 sized and configured to float in the water environment 12. A buoy cover 18 may be provided that is sized and configured to engage with the buoy hull 16 to generally form an enclosure. A chamber 20 is disposed within the buoy hull 16. The chamber 20 may be formed to be substantially airtight so as to provide buoyancy to the overall water sensor buoy 10 when place in the water environment 12. The overall design and configuration of the buoy hull 16 may be chosen from those which are well known to one of ordinary skill in the art. It is understood that the particular embodiment depicted of the buoy hull 16 and the use of a buoy cover 18 and resultant chamber 20 are exemplary in nature. Other floatation arrangements may be utilized that incorporate various materials, such as foam components, and use of inflatable air bladders for example.

As will be discussed in further detail below, the water sensor buoy 10 includes a sensor package 22 that is in removable engagement with the main buoy assembly 14. The sensor package 22 includes a sensor housing 24 that this in removable engagement with the buoy hull 16. The sensor package 22 further includes a water sensor 26 that is disposed in the sensor housing 24. The water sensor 26 has a sensor casing 28, a sensing element 30 attached to the sensor casing 28, and sensor electronics 30 in electrical communication with the sensing element 30. The sensor casing is attached to the sensor housing 24. In this regard the sensor casing 28 is structurally in communication and supported by the sensor housing 24 although other structural intermediate components may be utilized. In general, it is understood that the water sensor 26 may be provided as a unit, such as from a sensor manufacturer. The sensor casing 28 is understood to refer to the component that structurally supports the sensing element 30 and the sensor electronics 30 and the structural integrity of the water sensor 36.

The main buoy assembly 14 further includes a data processing and control unit 52, a data transmission unit 54, and a power source 56. The data processing and control unit 52 is supported by the buoy hull 16. The data processing and control unit 52 is sized and configured to generate a data signal upon receipt of sensor data from the water sensor 26. The data transmission unit 54 is supported by the buoy hull 16 and in electronic communication with the data processing and control unit 52. The data transmission unit 54 is sized and configured to wirelessly transmit a data signal generated by the data processing and control unit 52. The power source 56 is supported by the buoy hull 16 and being in electrical communication with the data processing and control unit 52 and the data transmission unit 54.

The water sensor 26 is in removable electronic communication with the data processing and control unit 52 and the power source 56. The water sensor 26 is sized and configured to sense an attribute of the water environment 12 and generate sensor data for receipt by the data processing and control unit 52. The water sensor 26 is electronically disengageable from the data processing and control unit 52 and the power source 56 upon the sensor housing 24 being disengaged from the buoy hull 16. The water sensor buoy further includes a watertight seal formed between the buoy hull 16 and sensor housing 24 with the sensor housing 24 engaged with the buoy hull 16. The water sensor buoy 10 further includes the electrical conduit 34 disposed through the watertight seal with the sensor housing 24 engaged with the buoy hull 16. The electrical conduit 34 is electrically connected to the sensor electronics 32. The electrical conduit 34 is electronically engageable with the data processing and control unit 52 and the power source 56 with the sensor housing 24 being engaged with the buoy hull 16. The electrical conduit 34 is electronically disengageable from the data processing and control unit 52 and the power source 56 upon the sensor housing 24 being disengaged from the buoy hull 16.

As mentioned above, the sensor package 22 is in removable engagement with the main buoy assembly 14. It is contemplated that the water sensor 26 must be maintained and serviced for a number of reasons, such as routine maintenance, cleaning, calibration and replacement. The end user of the water sensor buoy 10 is typically deployed in a large number of units, and not simply a single water sensor buoy 10. The maintenance and servicing of the water sensor buoy 10 would also not be done by the end user but rather by the supplier or vendor. As such, the water sensor buoys 10 would often need to be sent or transported to the facilities of the supplier or vendor. The buoy hull 16 accounts for the vast majority of the bulkiness, size or volume of the water sensor buoy 10. The cost of shipment and logistics of shipping of a large number of such water sensor buoys 10 may be significant. An aspect of the present invention recognizes that water sensor 26 is the specific component of the overall water sensor buoy 10 that may be of concern or in need of servicing. The water sensor buoy 10 as described above features the sensor package 22 which is separatable from the main buoy assembly 14 thereby readily allowing for the water sensor 26 to be removed from the buoy hull 16. In this regard the electrical conduit 34 is electronically disengageable from the data processing and control unit 52 and the power source 56 upon the sensor housing 24 being disengaged from the buoy hull 16. This is in contrast to prior art sensor buoy designs that may require laborious disassembly of various components, both structural and electronic, to remove the water sensor component. The end user would not be necessarily inclined or equipped to competently perform such disassembly.

The buoy hull 16 may include a buoy fitting 38. The sensor housing 24 may have a sensor cover 40 sized and configured to engage the buoy hull 16. A gasket 42 may be provided and disposed between the sensor housing 24 and the sensor cover 40. Hold-down screws 44 may be used to secure the sensor cover 40 and the gasket 42 to the sensor housing 24. The sensor cover 40 may be sized and configured to be circumferentially received by the buoy hull 16 for engagement of the buoy hull 16 and the sensor housing 24. In this regard, the sensor cover 40 may be sized and configured to be circumferentially received by the buoy fitting 38 for engagement of the buoy hull 16 and the sensor housing 24. The watertight seal may be disposed between the sensor cover 40 and the buoy hull 16 with the electrical conduit 34 is disposed through the sensor cover 40. The water sensor buoy 10 may further include an O-ring 48 disposed between the buoy hull 16, such as the buoy fitting 38, and the sensor housing 24 for forming the watertight seal. In the embodiment depicted, the O-ring 48 is circumferentially seated about the sensor housing 24. The chamber 20 may be watertight upon the buoy hull 16 being engaged with the sensor housing 24.

As will be discussed further below, according to another aspect of the invention, the water sensor buoy 10 may include a wiper wheel 64, a wheel motor 66, a water pump 68, and a wiper 112. The water sensor 26 is contemplated to have an exterior sensor surface 36 (as depicted in FIG. 8). The wiper wheel 64, the wheel motor 66, the water pump 68, and the wiper 112 may be used to for removal of particulates disposed upon the exterior sensor surface 36. The wiper wheel 64 and the wheel motor 66 may be supported by the sensor housing 24. The sensor package 22 may further include a pump plate 90 configured to support the water pump 68. A spacer 94 may be used to vertically offset the sensor housing 24 from the pump plate 90. A spacer fastener 96 is axially received in the spacer 94 to attach the sensor housing 24 to the pump plate 90. The sensor package 22 may further include a housing plate 92 that is attached to the pump plate 90 using a housing fastener 98. A sensor guard 46 is provided that generally surrounds the lateral entirety of the sensor package 22 with the housing plate 92 on one end and the sensor cover 40 on the other end. The sensor guard 46 includes various openings to allow for the free flow of water from the water environment 12 to enter and exit the sensor package 22 to allow for the water sensor 26 to make accurate measurements of the desired attributes of the water environment being sensed by the water sensor 26. At the same time, the sensor guard 46 provides a degree of physical protection for the components within the sensor package 22.

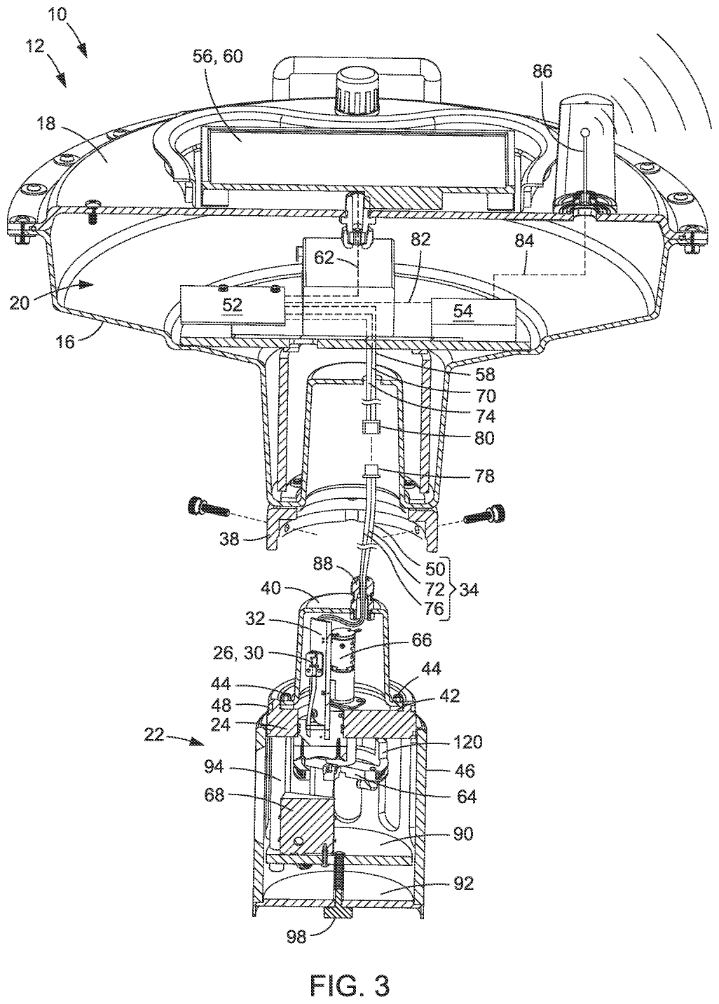

With particular reference to FIG. 3, there is symbolically depicted the various electronic components and related electronic connections of the water sensor buoy 10. The power source 56 is connected to the data processing and control using 52 via buoy power wiring 62. A solar array 60 may be provided to generate electricity for the power source 56. The power source 56 may include a battery (not shown). The sensor housing 24 may have an exit port 88. The electrical conduit 34 is disposed through the exit port 88. The electrical conduit 34 is electrically connectable to the data processing and control unit 52 and the power source 56 though the entry port 88.

As mentioned above, the electrical conduit 34 is electrically connected to the sensor electronics 32. In this regard the electrical conduit 34 may include sensor wiring 50 that is connected to the sensor electronics 32 of the water sensor 26. The electrical conduit 34 may further include motor wiring 72 that is connected to the wheel motor 66 and pump wiring 76 that is connected to the water pump 68. The electrical conduit 34 may terminate in an exit port coupling 78.

An entry port coupling 80 may be provided that is sized and configured to engage and electronically connect with the exit port coupling 78 to allow the electrical conduit 34 to be readily electrically engaged and disengaged from the electronic components of the main buoy assembly 14. The data processing and control unit 52 may thus be connected to the entry port coupling 80 using the buoy sensor wiring 58, the buoy motor wiring 70, and the buoy pump wiring 74. The entry port coupling 80 and the complementary exit port coupling 78 are sized and configured to allow for electrical connections of the sensor wiring 50 to the buoy sensor wiring 58, the motor wiring 72 to the buoy motor wring 70, and the pump wiring 76 to the buoy pump wiring 74. It is contemplated that the provision of power from the power source 56 may be supplied to the water sensor 26, the wheel motor 66 and the water pump 68 via power supplied through the data processing and control unit 52. The electrical conduit 34 is electronically engageable with the data processing and control unit 52 and the power source 56 with the sensor housing 24 being engaged with the buoy hull 16. As such, the electrical conduit 34 may be readily connected to the main buoy assembly 14 upon the buoy hull 16 being physically engaged with the sensor housing 24, such as by a simple connection of the exit port coupling 78 to the entry port coupling 80. The electrical conduit 34 is electronically disengageable from the data processing and control unit 52 and the power source 56 upon the sensor housing 24 being disengaged from the buoy hull 16. Similarly, the electrical conduit 34 may be readily disengaged from the main buoy assembly 14 upon the buoy hull 16 being physically engaged with the sensor housing 24, such as by a simple disconnection of the exit port coupling 78 with the entry port coupling 80.

As mentioned above, the water sensor 26 includes the sensing element 30 that is sized and configured to sense an attribute of the water environment 12 and generate sensor data for receipt by the data processing and control unit 52. The water sensor 26 may be any number of sensors used to detect or sense a physical property of the water or liquid in which the water sensor buoy 10 is deployed. The water sensor 26 may be chosen from those which are well known to one of ordinary skill in the art. For example, the water sensor 26 may be optical fluorescence sensors, acoustic backscatter sensors, photosynthetically active radiation (PAR) sensors, conductivity sensors, hydrophones, and pH sensors. Some general descriptions are below.

Optical fluorescence sensors are single wavelength in situ fluorescence and turbidity probes used for monitoring various water quality parameters such as chlorophyll-a concentration and turbidity in aquatic systems. The probes consist of a light source and a detector that measure the intensity of the emitted fluorescence and scattered light, respectively. The probe is submerged in the water and emits light at a specific wavelength through a clear face, such as the exterior sensor surface 36, which excites chlorophyll-a and other fluorescent compounds in the water. The emitted fluorescence is then detected by a detector of the probe through the same clear face and measured in terms of its intensity. Turbidity, which is a measure of the amount of suspended particles in the water, is measured by the detector of the probe through the detection of scattered light. The data obtained from these probes can be used for various environmental monitoring applications, such as assessing the health of aquatic ecosystems, detecting harmful algal blooms, and monitoring water treatment processes.

Acoustic backscatter sensors are used to measure the concentration and size distribution of suspended particles or sediments in water. These sensors work by emitting an acoustic signal, typically in the range of 1 to 10 MHz, and measuring the echo or backscatter of the signal as it interacts with particles in the water. These sensors typically consist of a transducer that emits the acoustic signal and receives the backscatter of the signal, and a signal processing unit that converts the received signal into data on the concentration and size distribution of the particles. The transducer emits a short pulse of sound waves that travel through the water and interact with the particles. Some of the sound waves are scattered back toward the transducer, and these echoes are received and processed by the signal processing unit. The strength of the backscatter signal is related to the concentration and size of the particles in the water. Relatively larger and denser particles will produce a stronger backscatter signal than smaller and less dense particles. By analyzing the backscatter signal, the sensor can be used to determine the concentration and size distribution of particles in the water. Acoustic backscatter sensors are used in a variety of applications, such as monitoring sediment transport in rivers and coastal environments, assessing the quality of drinking water, and studying the behavior of plankton and other suspended particles in the ocean or other water environment.

Photosynthetically active radiation (PAR) sensors work by measuring the intensity of light in the wavelength range of 400 to 700 nanometers, which corresponds to the range of light that is used by plants for photosynthesis. These sensors typically consist of a photodiode or photovoltaic cell that is sensitive to light in this range and a signal processing unit that converts the light intensity into an electrical signal that can be recorded or analyzed. When light hits the photodiode or photovoltaic cell in the sensor, it generates a small electric current that is proportional to the intensity of the light. The signal processing unit amplifies the electric current and converts it into a voltage signal that can be recorded or transmitted to other devices for further analysis.

There are a variety of types of conductivity sensors available that are used for measuring the electrical conductivity of water, which is related to the concentration of dissolved materials in the water, such as salts. These sensors include inductive conductivity sensors, four-electrode conductivity sensors, contacting conductivity sensors, toroidal conductivity sensors and optical conductivity sensors. Inductive conductivity sensors measure the electrical conductivity of water using an inductive coil and an electrode system. The inductive coil generates a magnetic field, which induces an electrical current in the water. The current is then measured by the electrode system, which is proportional to the conductivity of the water. Four-electrode conductivity sensors measure the conductivity of water using four electrodes. Two of the electrodes are used to apply an AC current to the water, while the other two electrodes measure the voltage drop across the water. By measuring the voltage drop, the conductivity of the water can be calculated. Contacting conductivity sensors measure the conductivity of water using two electrodes that are in contact with the water. The electrical resistance between the electrodes is measured, and the conductivity of the water is calculated using Ohm's Law. Toroidal conductivity sensors measure the conductivity of water using a toroidal coil that surrounds the water. The coil generates a magnetic field, which induces an electrical current in the water. The current is then measured, and the conductivity of the water is calculated. Optical conductivity sensors measure the conductivity of water using an optical method, where light is passed through the water and the absorption of the light is measured. The absorption is related to the conductivity of the water.

Hydrophones are underwater microphones that are used to detect and measure sound waves in water. These sensor work by converting sound waves into electrical signals that can be recorded or analyzed. Hydrophones consist of a piezoelectric element that is housed in a cylindrical or spherical case. The piezoelectric element is made of a material that generates an electrical signal when subjected to pressure or vibration. When sound waves travel through water, they create variations in pressure that cause the piezoelectric element to vibrate. The vibrations generate electrical signals that are proportional to the sound wave's amplitude and frequency. Hydrophones can be used for a wide range of applications, such as oceanography, marine biology, underwater communications, and military sonar systems. The sensitivity and frequency response of a hydrophone depend on several factors, such as the size and shape of the piezoelectric element, the acoustic properties of the surrounding water, and the design of the hydrophone housing.

There are a variety of pH sensors for measuring the acidity or alkalinity of a solution. These include glass electrode pH sensors, ion-selective field-effect transistor (ISFET) pH sensors, optical pH sensors, conductivity-based pH sensors, and microelectromechanical system (MEMS) pH sensors. Glass electrode pH sensors are the most common type of pH sensors. They consist of a glass membrane that is sensitive to changes in pH and an electrode that measures the potential difference between the sample solution and a reference solution. Ion-selective field-effect transistor (ISFET) pH sensors use a thin film transistor and a gate electrode that is sensitive to changes in pH. The gate electrode is covered with a pH-sensitive material that interacts with the sample solution, causing a change in the transistor's electrical properties. Optical pH sensors use fluorescent dyes or indicators that change their optical properties in response to changes in pH. The changes in the fluorescence can be measured and used to determine the pH of the sample solution. Conductivity-based pH sensors use the principle of conductivity to measure changes in pH. The conductivity of a solution is affected by changes in pH, and the changes in conductivity can be used to determine the pH of the solution. Microelectromechanical system (MEMS) pH sensors use microfabrication techniques to create tiny mechanical structures that can measure changes in pH. The mechanical structures are coated with a pH-sensitive material, and changes in the mechanical properties can be used to determine the pH of the sample solution. Overall, each type of pH sensor has its own advantages and limitations, and the choice of sensor depends on the specific application and the accuracy required for the measurement.

The power source 56, the data processing and control unit 52, the data transmission unit 54, the water sensor 26, the water pump 68, and the wheel motor 66, and the associated circuitry, wiring and interconnections may be chosen from any of those designs and arrangements which are well known in one of ordinary skill in the art. While the various components are depicted as being separate components it is understood that various ones of the components may be combined in a single unit. Similarly, while the data processing and control unit 52 is depicted as a single unit, it is contemplated the data processing tasks associated with the receipt and processing of the sensor data may be handled by an electrical component that is separate from the control circuitry that controls the rotation of the wiper wheel 64 and the water pump 68. In addition, it is contemplated that the water sensor buoy 10 may include other electrical components such as GPS location hardware/software and other or additional water sensors or environmental sensors (such as for measuring water temperature, air temperature and pressure, surface wind, etc.).

The data processing and control unit 52 is configured to process the sensor data or data signals generated by the sensor electronics 32 as may be desired for transmission by the data transmission unit 54. It is also contemplated that the sensor electronics 32 may perform some degree of data pre-processing prior to being sent to the data processing and control unit 52. The data processing and control unit 52 generally is configured to control the operation of the other on-board electronics with data and control signals as well as power distribution. The data processing and control unit 52 may feature other functions, such as running system diagnostics for generating error/fault/status data associated with any of the on-board electronics.

As mentioned above, the data transmission unit 54 is sized and configured to wirelessly transmit a data signal generated by the data processing and control unit 52. Wiring 82 is used to send data signals from the data processing and control unit 52 to the data transmission unit 54. The data transmission unit 54 may be in electrical communication with the power source 56 via the wiring 82 and the buoy power wiring 62. The data transmission unit 54 is connected to an antenna 86 via wiring 84 for wirelessly transmitting the data signal. In this regard, the user of the water sensor buoy 10 may remotely monitor the desired sensed water attribute as sensed by the water sensor 26. It is contemplated that the data transmission unit 54 may additionally be used to transmit any other data signals generated by the data processing and control unit 52, such as other sensor data, error/fault/status data associated with any of the on-board electronics, GPS location data and the like. Further, the data transmission unit 54

According to another aspect of the invention, there is provided a method of using a water sensor buoy 10 for use in the water environment 12. The method includes the step of floating the water sensor buoy 10 in the water environment 12. The method may further include electronically disengaging the electrical conduit 34 from the data processing and control unit 52 and the power source 56.

According to an aspect of the invention, there is provided the water sensor buoy 10 for use in the water environment 12. The water sensor includes the sensing element 30 and the exterior sensor surface 36 facing away from the sensing element 30. The exterior sensor surface 36 is exposed to the water environment 12 upon the water sensor buoy 10 being placed in the water environment 12. The exterior sensor surface 36 refers to that surface which is exposed to the water environment 12 which the water sensor 26 is sensing. The water sensor 26 may be provided by a sensor manufacturer or vendor. However, depending upon the nature of the particular sensor type, it is contemplated that additional protective lenses or layer, such as an additional glass layer or pane. The water sensor 26 may include a sensor foil with the exterior sensor surface 36 being formed on the sensor foil. The sensor foil may be formed of glass. The sensor foil may be translucent.

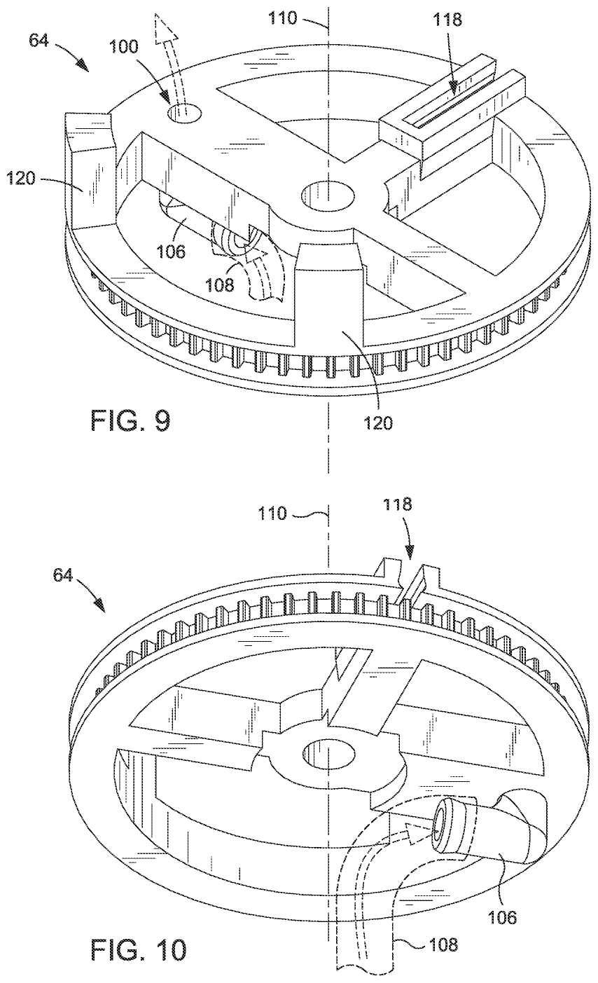

Referring additionally to FIGS. 9 and 10, the water sensor buoy 10 further includes the wiper wheel 64 rotatably supported by the buoy hull 16. The water sensor buoy 10 further includes the wiper 112 attached to the wiper wheel 64. The wiper 112 is sized and configured to slidably engage the exterior sensor surface 36 upon rotation of the wiper wheel 64 for removal of particulates disposed upon the exterior sensor surface 36. The wiper 112 is thus arranged to wipe off or brush away unwanted material accumulation that may be present upon the exterior sensor surface. The water sensor buoy 10 further includes the water pump 68 being in electrical communication with the power source 56. The water sensor buoy 10 further includes a water jet outlet 100 in fluid communication with the water pump 68. The water jet outlet 100 is sized and configured to direct a water stream at the exterior sensor surface 36 for removal of particulates disposed upon the exterior sensor surface 36.

Fouling is an issue associated with all sensor instrumentation deployed in water environments that causes a loss in sensitivity and reproducibility of the water sensor data, thus requiring frequent re-calibration and/or producing erroneous results. In particular, the exterior sensor surface 36 may become fouled with accumulation particulates disposed upon the exterior sensor surface 36. This may be due to adhesion or deposition of material in the water environment 12 and growth of algae and other living organisms. An aspect of the invention recognizes that the wiper wheel 64 with the integrated wiper 112 and water jet outlet 100 is uniquely configured to effectively remove particulates disposed upon the exterior sensor surface 36. In this regard the wiper wheel 64 may be considered an anti-fouling wheel.

According to various embodiments, the water sensor buoy 10 may further include the wheel motor 66 electrically connected to the power source 56 and engaged with the wiper wheel 64. The wheel motor 66 is sized and configured to rotate the wiper wheel 64 to move the wiper 112 relative to the exterior sensor surface 36. The wiper wheel 64 may include a radially extending slot 118, and the wiper 112 is disposed in the slot 118. The wiper 112 may be press-fit attached to the wiper wheel 64 in the slot 118. In this respect the wiper 112 may be readily removed and replaced as needed. The wiper 112 may include a brush 114. The wiper 112 may further include a wiper arm 116 and the brush 114 extends from the wiper arm 116. The wiper arm 116 may be disposed in the slot 118.

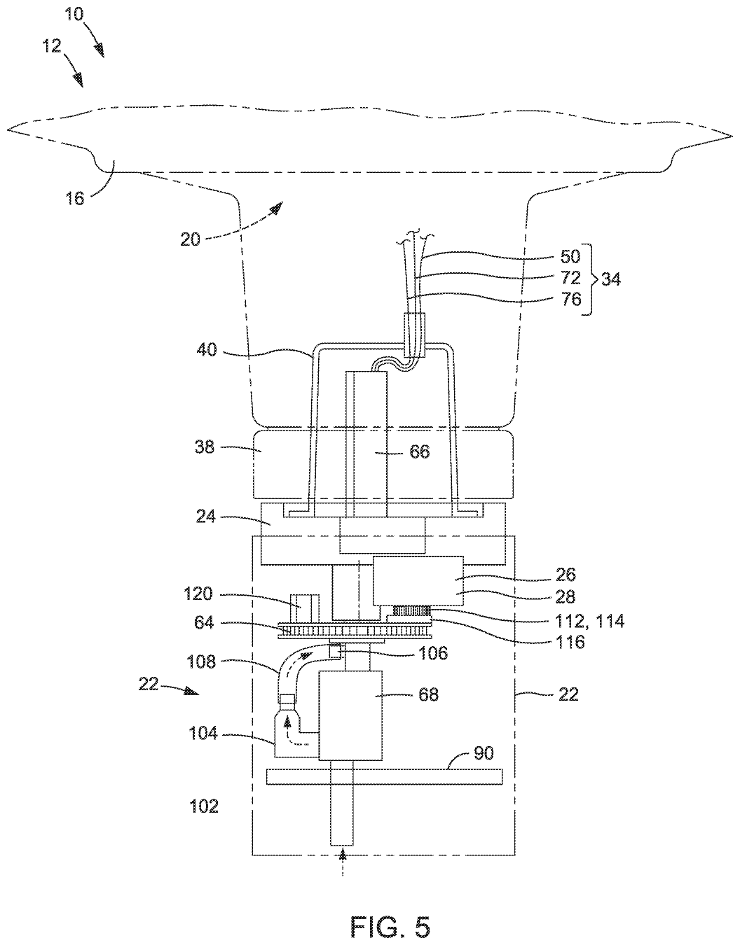

The water pump 68 may include a pump inlet 102. The pump inlet 102 is configured to draw in fluid from the water environment 12. The water pump 68 may further include a wheel inlet 106. The wiper wheel 64 may include a wheel inlet 106. The pump outlet 106 may be fluidically connected to the wheel inlet 106 via tubing 108 (as indicated in dashed lining in FIGS. 5, 9 and 10, and not show in other figures for ease of depiction of other components). The water pump 68 is configured to pump water from water environment 12 through the tubing 108 to the pump inlet 102. The wiper wheel 64 may include an internal fluid passage and the water jet outlet 100 may be in fluid communication with the wheel inlet 106 for providing water flow from the water pump 68 out the water jet outlet 100. It is contemplated that the water jet outlet 100 may include a nozzle (not shown) to provide any desired flow velocity or spay pattern as desired.

The wiper wheel 64 is configured to rotate about a wheel axis of rotation 110. The whole motor 66 is controlled by the data processing and control unit 52 to rotate the wiper wheel 64 in a back and forth motion to selectively position the wiper 112 and the water jet outlet 110 adjacent the exterior sensor surface 36.

The wiper wheel 64 may include stoppers 120. The stoppers 120 may be positioned so as to limit the range of motion or angle of rotation of the wiper wheel 64. The stoppers 120 may be configured to come into physical contact with the sensor casing 28 to prevent excessive rotation of the wiper wheel 64. It is contemplated that the wiper wheel 64 need only rotate enough so as to allow the wiper 112 to sweep across the exterior sensor surface 36 and the water jet outlet 100 to also sweep across the exterior sensor surface 36 to perform the removal operations.

According to another aspect of the invention, there is provided a method of operating a water sensor buoy for use in a water environment. The method includes the steps of floating the water sensor buoy 10 in the water environment 12. The method further includes the step of rotating the wiper wheel 64 to move the wiper 112 to slidably engage the exterior sensor surface 36 for removal of particulates disposed upon the exterior sensor surface 36. The method further includes the step of directing a water stream from the water jet outlet 100 at the exterior sensor surface 36 for removal of particulates disposed upon the exterior sensor surface 36.

According to various embodiments, the method may further include pumping a fluid from the water environment 12 by the water pump 68 to the water jet outlet 100. The method may further include generating a data signal by the data processing and control unit 52 upon receipt of sensor data from the water sensor 26. The method may further include using the data transmission unit 54 to wirelessly transmit the data signal generated by the data processing and control unit 52. The wiper may include the brush 114, and the rotating of the wiper wheel 64 may include slidably engaging the brush 114 against the exterior sensor surface 36 for removal of particulates disposed upon the exterior sensor surface 36.

The particulars shown herein are by way of example only for purposes of illustrative discussion, and are presented in the cause of providing what is believed to be the most useful and readily understood description of the principles and conceptual aspects of the various embodiments set forth in the present disclosure. In this regard, no attempt is made to show any more detail than is necessary for a fundamental understanding of the different features of the various embodiments, the description taken with the drawings making apparent to those skilled in the art how these may be implemented in practice.

What is claimed is:

1. A water sensor buoy for use in a water environment, the water sensor buoy comprising:
a main buoy assembly sized and configured to float in a water environment, the main buoy assembly including:
a buoy hull sized and configured to float in the water environment;
a data processing and control unit supported by the buoy hull, the data processing and control unit sized and configured to generate a data signal upon receipt of sensor data from a water sensor;
a data transmission unit supported by the buoy hull and in electronic communication with the data processing and control unit, the data transmission unit being sized and configured to wirelessly transmit a data signal generated by the data processing and control unit; and
a power source supported by the buoy hull and being in electrical communication with the data processing and control unit and the data transmission unit;
a sensor package being in removable engagement with the main buoy assembly, the sensor package including:
a sensor housing in removable engagement with the buoy hull; and
a water sensor disposed in the sensor housing, the water sensor having a sensor casing, a sensing element attached to the sensor casing, and sensor electronics in electrical communication with the sensing element, the sensor casing being attached to the sensor housing, the water sensor being in removable electronic communication with the data processing and control unit and the power source, the water sensor being sized and configured to sense an attribute of the water environment and generate sensor data for receipt by the data processing and control unit, the water sensor being electronically disengageable from the data processing and control unit and the power source upon the sensor housing being disengaged from the buoy hull;
a watertight seal formed between the buoy hull and sensor housing with the sensor housing engaged with the buoy hull; and
an electrical conduit disposed through the watertight seal with the sensor housing engaged with the buoy hull, the electrical conduit being electrically connected to the sensor electronics, the electrical conduit being electronically engageable with the data processing and control unit and the power source with the sensor housing being engaged with the buoy hull, the electrical conduit being electronically disengageable from the data processing and control unit and the power source upon the sensor housing being disengaged from the buoy hull.

2. The water sensor buoy of claim 1 further includes an O-ring disposed between the buoy hull and the sensor housing for forming the watertight seal.

3. The water sensor buoy of claim 1 wherein the sensor housing has a sensor cover sized and configured to engage the buoy hull, the watertight seal is disposed between the sensor cover and the buoy hull, the electrical conduit is disposed through the sensor cover.

4. The water sensor buoy of claim 3 wherein the sensor cover is sized and configured to be circumferentially received by the buoy hull for engagement of the buoy hull and the sensor housing.

5. The water sensor buoy of claim 1 wherein the sensor housing has an exit port, the electrical conduit being disposed through the exit port.

6. The water sensor buoy of claim 1 wherein the electronic conduit includes an exit port coupling sized and configured to electronically engage and disengage with the data processing and control unit and the power source.

7. The water sensor buoy of claim 1 wherein the buoy hull includes a chamber, the chamber is watertight upon the buoy hull being engaged with the sensor housing.

8. The water sensor buoy of claim 1 wherein the water sensor is a pH sensor.

9. The water sensor buoy of claim 1 wherein the water sensor is an optical fluorescence sensor.

10. The water sensor buoy of claim 1 wherein the water sensor is a hydrophone.

11. A method of using a water sensor buoy for use in a water environment, the method comprising the steps of:
a) floating the water sensor buoy in the water environment, the water sensor buoy including:
a main buoy assembly sized and configured to float in a water environment, the main buoy assembly including:
a buoy hull sized and configured to float in the water environment;
a data processing and control unit supported by the buoy hull, the data processing and control unit sized and configured to generate a data signal upon receipt of sensor data from a water sensor;
a data transmission unit supported by the buoy hull and in electronic communication with the data processing and control unit, the data transmission unit being sized and configured to wirelessly transmit a data signal generated by the data processing and control unit; and
a power source supported by the buoy hull and being in electrical communication with the data processing and control unit and the data transmission unit;
a sensor package being in removable engagement with the main buoy assembly, the sensor package including:
a sensor housing in removable engagement with the buoy hull; and
a water sensor disposed in the sensor housing, the water sensor having a sensor casing, a sensing element attached to the sensor casing, and sensor electronics in electrical communication with the sensing element, the sensor casing being attached to the sensor housing, the water sensor being in removable electronic communication with the data processing and control unit and the power source, the water sensor being sized and configured to sense an attribute of the water environment and generate sensor data for receipt by the data processing and control unit, the water sensor being electronically disengageable from the data processing and control unit and the power source upon the sensor housing being disengaged from the buoy hull;
a watertight seal formed between the buoy hull and sensor housing with the sensor housing engaged with the buoy hull; and
an electrical conduit disposed through the watertight seal with the sensor housing engaged with the buoy hull, the electrical conduit being electrically connected to the sensor electronics, the electrical conduit being electronically engageable with the data processing and control unit and the power source with the sensor housing being engaged with the buoy hull, the electrical conduit being electronically disengageable from the data processing and control unit and the power source upon the sensor housing being disengaged from the buoy hull;
b) removing the water sensor buoy from the water environment;
c) disengaging the sensor housing from the buoy hull; and
d) electronically disengaging the electrical conduit from the data processing and control unit and the power source.
